(19) United States
(12) Patent Application Publication
(10) Pub. No.: US 2019/0008692 A1
(43) Pub. Date: Jan. 10, 2019

(54) DRESSING ARRANGEMENT AND METHOD FOR TREATING A WOUND

(71) Applicant: SEFAR AG, Heiden (CH)

(72) Inventors: Gerd GERDES, Abtwil (CH); Jérémie WEBER, Klaus (AT)

(73) Assignee: SEFAR AG, Heiden (CH)

(21) Appl. No.: 16/068,296

(22) PCT Filed: Nov. 21, 2016
(86) PCT No.: PCT/EP2016/078294

§ 371 Date: Jul. 5, 2018

(30) Foreign Application Priority Data
Jan. 11, 2016 (EP) 16150730.6

(51) Int. Cl. A61F 13/02; A61M 1/00

(52) U.S. Cl. CPC A61F 13/0206; A61F 13/0253; A61M 1/0088; A61F 13/0216

(57) ABSTRACT

The invention relates to a dressing arrangement and to a method for treating a wound of a human or animal body by means of vacuum. The dressing arrangement has an absorbent body for absorbing wound secretions, the absorbent body being designed for insertion in a cavity of the wound. Furthermore, a separating element is provided, which is arranged between the wound and the absorbent body. Furthermore, a cover element, which is designed to cover the wound, together with the absorbent body, in a substantially airtight manner, and a connecting means are provided, which connecting means is arranged for connecting a vacuum source to the cover element. According to the invention, it is provided that the separating element is designed to be larger than the wound to be treated, a protruding edge portion being formed, and that the edge portion is attached to a skin region at the edge of the wound.

The invention relates to a dressing arrangement for treating a wound of a human or animal body by means of vacuum, comprising an absorbent body for absorbing wound secretions, the absorbent body being designed to be inserted in a cavity of the wound, a separating element, which is arranged between the wound and the absorbent body, a cover element, which is designed to cover the wound, together with the absorbent body, in a substantially airtight manner, and a connecting means, which is arranged for connecting a vacuum source to the cover element.

WO 2007/030598 A2 discloses a generic dressing arrangement and a method for treating a wound of a human or animal body, an absorbent body being inserted in a cavity of the wound to absorb wound secretions, a separating element being arranged between the wound and the absorbent body, the wound, together with the inserted absorbent body, being covered by means of a cover element in a substantially airtight manner, and a vacuum source being connected to the cover element by a connecting means, by means of which source a vacuum in the covered wound is adjusted. As a separating element, a gauze, that is to say a coarse-meshed woven cotton fabric, is used.

An additional dressing arrangement of this type and a method of this type are disclosed for example in WO 2006/048246 A1. In this known vacuum treatment, in the case of wounds having relatively large cavities, wound secretions can be reliably conducted away without being able to cause lasting damage to the healthy tissue in the process. The vacuum causes wound secretions to permeate the absorbent body. The wound secretions can be absorbed therein or conducted away as a whole out of the absorbent body and the covered wound by means of a discharge line.

It is known that the absorbent body is conventionally formed of a foam material. This foam material is cut directly to the size of the wound so that the absorbent body fills the cavity of the wound substantially so as to fit to said wound, but does not come into contact with the adjacent skin regions. The absorbent body formed of foam material is conventionally designed with a certain distance from the edge of the wound in order to prevent irritation of the skin by the foam material and by wound secretion absorbed in the absorbent body.

Furthermore, it is known to provide a film element as a separating layer between the wound and the absorbent body to prevent the absorbent body from being incorporated into the tissue. A corresponding dressing arrangement is disclosed in US 2008/0177253 A1.

GB 2468905 A describes a dressing material for a vacuum method, in which the absorbent body is produced from a three-dimensional knitted fabric. In addition, the three-dimensional knitted fabric can be encased in a perforated plastics material sheet or a non-woven material.

EP 2 959 871 A1 discloses a dressing product which is mounted in a sealed chamber.

The object addressed by the invention is that of indicating a method and an arrangement for treating a wound which allow particularly gentle wound treatment.

The dressing arrangement according to the invention is characterized in that the separating element is designed to be larger than the wound to be treated, a protruding edge portion being formed, and in that the edge portion is attached to a skin region at the edge of the wound.

A basic concept of the invention can be considered to be that the separating element provided to prevent the absorbent body from being incorporated is formed so as to be specifically larger than the absorbent body and the wound together so that the surrounding skin region at the edge of the wound can also be covered thereby. Thus, particularly good protection of the wound against contamination and of the edge of the wound against irritation is achieved.

According to one finding of the invention, the absorbent body is conventionally cut to the required size directly in the vicinity of the wound by the health-care staff. In so doing, the foam material of the absorbent body is held directly over the wound and trimmed using scissors. In this case, there is a risk that smaller particles of the absorbent body could enter the wound and lead to damage, inflammation or pain for the patient.

Furthermore, there is a risk that the absorbent body could come into direct contact with the edge of the wound and the adjacent skin region in an undesirable manner. This can lead to irritation and what is known as maceration of the affected region of tissue. This makes it harder and more complicated for the wound to heal. By means of the separating element, which also covers the edge of the wound, this is prevented, even if the absorbent bodies protrude out of the wound cavity.

According to the invention, a separating element is initially applied to a large surface area of the wound, a skin region at the edge of the wound also being covered and protected by a protruding edge portion. Thus, by cutting the absorbent body to size, protection against contamination or against undesirable contact with the wound can be achieved. In this case, an absorbent element which protrudes beyond the edge of the wound can preferably also be used. Preformed standard absorbent elements can thus be applied without being cut to size.

According to the invention, it is provided that, at the edge portion, an adhesive layer is arranged at least in some regions to adhere the separating element to the skin region. The adhesive layer can preferably have a dermatological acrylic or silicone adhesive. According to the invention, it is preferable for the separating element to have a rectangular or elongate shape, a total of two strip-like adhesive layers extending along the two long sides of the separating element. In this case, the separating element does not have to be adhered around the edge of the wound on all sides, but rather can be fixed to only two opposing sides of the wound by means of the adhesive layer. The adhesive layer ensures simple and secure handling.

In principle, the separating element can be produced from any type of material. According to one embodiment of the invention, it is particularly advantageous for the separating element to have a woven fabric. A woven fabric is particularly permeable to air and gentle on wounds.

According to one development of the invention, a particularly effective separating element is achieved in that the woven fabric is formed of mono-filament threads having a smooth surface and preferably a thread diameter of between 10 and 100 μm, in particular between 30 and 50 μm. In this case, the mono-filament threads can be produced from polyamide, polyester, polypropylene or another suitable plastics material. It has become apparent that polyester (PET) is particularly gentle on wounds and counteracts growing together. A possible explanation can be seen in the fact that PET mono-filament threads have a particularly smooth surface, which counteracts adhesion of proteins, human cells and tissues.

According to one development of the invention, it is preferable for the separating element to be designed to be bio-compatible and anti-adhesive. This can be achieved in that the mono-filament threads are provided with a corresponding bio-compatible or anti-adhesive coating, for example PTFE, or the separating element as a whole is provided with such a layer. By means of a corresponding material selection and/or coating, a non-sensitizing or hypoallergenic property can additionally be adjusted.

A particularly good effect of the separating element is achieved according to one embodiment of the invention in that the woven fabric is provided with a mesh size of from 40 to 500 μm, preferably 70 to 300 μm. In this case, the mesh size relates to woven fabric and also to pore sizes, if membranes or perforated films are used. In the case of a woven fabric, preferably a single woven fabric, in particular having a plain, twill or satin weave, is preferably provided. The thickness of the woven fabric is between 20 and 200 μm, preferably between 50 and 100 μm.

According to another embodiment of the invention, it is preferable for the cover element to be designed to be larger than the separating element. The cover element is in particular an airtight film which can optionally be reinforced by a woven fabric material. The cover element thus covers not only the wound but also the separating element as a whole. On the cover element, a connection valve is provided, by means of which a vacuum source can be connected. By means of the vacuum source, for example a pump, a vacuum can be adjusted underneath the cover element in the region of the wound. As a result, wound secretions are conducted away from the damaged tissue into the absorbent body through the separating element. The wound secretions can be conducted away from the absorbent element to the outside via the connection valve.

The invention further relates to a separating element for the dressing arrangement according to the invention, wherein the separating element is detachably arranged on a carrier element. In this case, the carrier element is a thin plastics material sheet or a sheet of paper to which the separating element is initially detachably attached. This allows reliable handling of the thin separating element.

According to one development, it is particularly preferable for the carrier element to be a carrier film which can be cut to size. Thus, the carrier element, together with the carrier film, can be cut to the size of the wound. This makes it possible to cut the thin and thus very sensitive separating element to size very precisely. The separating element is removed from the carrier film only immediately before applying the separating element to the wound region.

A further improvement in handling is achieved according to one embodiment according to the invention in that the separating element and/or the carrier film are transparent. The arrangement consisting of the separating element and the carrier film can thus be held directly on the wound region and cut specifically to the required size there.

Furthermore, it is particularly expedient for the separating element to be detachably attached on the carrier element by an adhesive layer which is designed for fixing the separating element to a skin region. The adhesive layer, which preferably consists of two opposing adhesive strips along the long edges of the separating element, can thus fulfill a dual function, namely fixing to the carrier element and fixing to the skin region.

The method according to the invention for treating a wound is characterized in that the separating element is designed to be larger than the wound to be treated, a protruding edge portion being formed, and in that the edge portion is attached to a skin region at the edge of the wound. The method according to the invention is carried out in particular using a dressing arrangement of the above-described type. The above-described advantages are achieved accordingly.

A particularly good handling is achieved according to the method according to the invention in that the separating element is adhered to the edge portion on the skin region by an adhesive layer. In this case, the adhesive layer is preferably arranged along two edge sides of the separating element. As a result, a reliable, but not too tight, attachment of the separating element to the wound can be achieved.

A further preferred embodiment of the method according to the invention consists in the fact that the separating element is detachably attached to a carrier element which can be cut to size and, before application to the wound, is cut to the intended size together with the carrier element. This allows very good handling and an accurately fitting cut of the thin and sensitive carrier element, together with the stable carrier element. The carrier element can be a sheet of paper, a woven fabric or preferably a plastics material sheet.

The invention will be described further hereinafter by way of preferred embodiments, which are shown schematically in the drawings, in which:

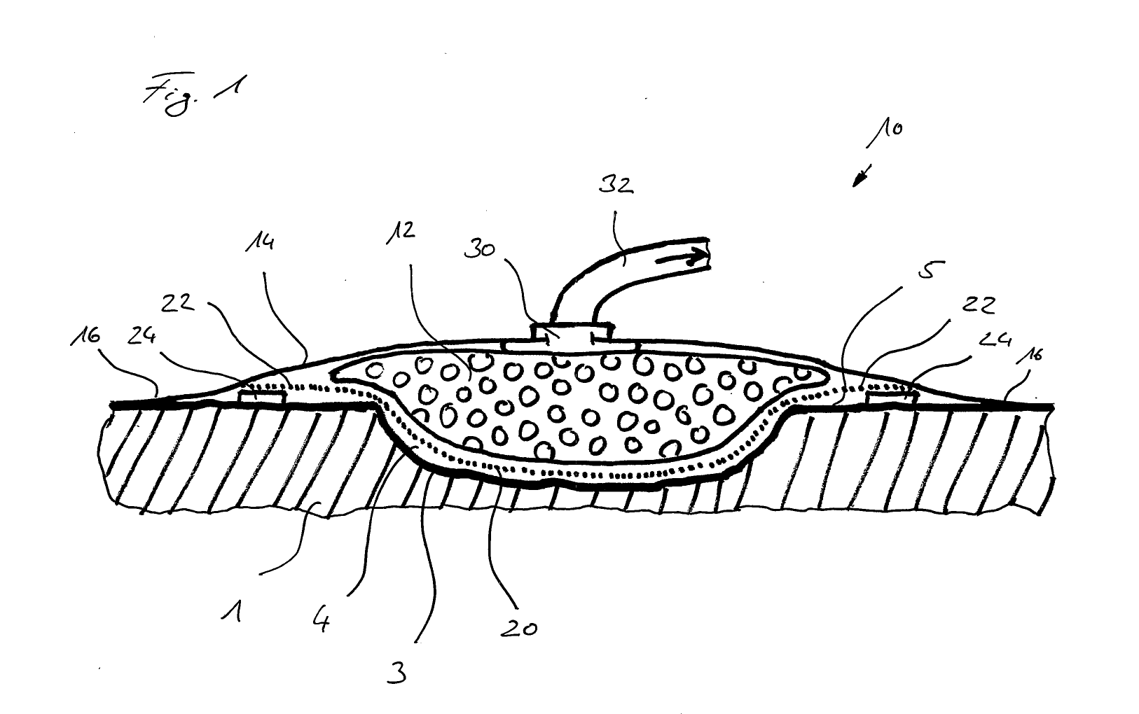

FIG. 1 is a schematic cross-sectional view of a dressing arrangement according to the invention; and

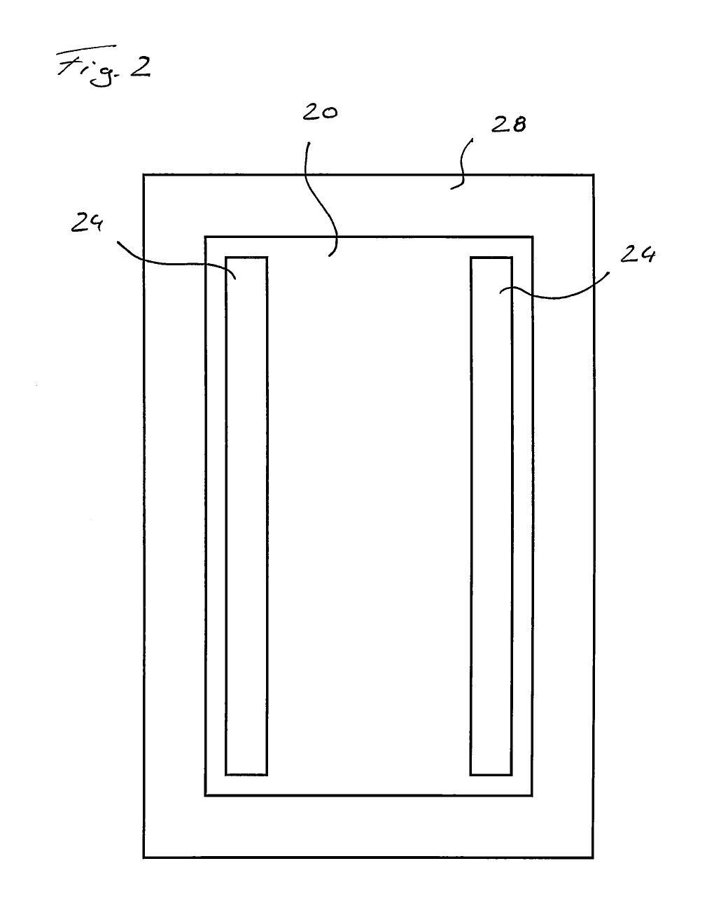

FIG. 2 is a schematic plan view of a separating element according to the invention.

FIG. 1 shows a wound 3 in a body 1 of a human or an animal having a cavity 4. In this case, the cavity 4 is filled substantially by an inserted absorbent body 12 made of a foam material. The absorbent body 12 is conventionally cut to size individually according to the size of the wound.

In order to prevent the absorbent body 12 from being incorporated into the wound 3, a separating element 20 made of a woven fabric is arranged between the absorbent body 12 and the wound 3. According to the invention, the thin separating element 20 having lateral edge portions 22 extends beyond the actual region of the wound 3, and said element is fixed to a skin region of the body 1 by means of two strip-shaped adhesive layers 24 which are at a distance from one another. The separating element 20 is laid in the wound 3 before the absorbent body 12 is inserted into the cavity 4. The absorbent body 12 can thus be cut to size directly on the wound 3 in a safe manner. Small particles of waste material which are produced when cutting the foam material of the absorbent body 12 to size can thus be prevented from directly entering the wound 3 by the separating element 20.

Edge portions 22 of the separating element 20 protrude beyond the wound 3 in a defined manner, the separating element 20 being able to be attached to the skin region 5 of the body 1 by means of adhesive strips 24 on the lower face of the edge portions 22. Through the protruding edge portions 22 also form additional protection for the skin region 5 along the edge of the wound 3. As a result, the absorbent body 12 made of the foam material can also protrude out of the wound 3 into the skin region 5 without the foam material causing noticeable irritation to the skin or harmful maceration. This also makes it possible to insert substantially pre-cut absorbent bodies 12 into wounds 3 without individually cutting to size.

The absorbent body 12 and the separating element 20 protruding in a defined manner with respect to the wound 3 and the absorbent body 12 are covered by a film-type cover element 14. The cover element 14 is attached to the skin of the body 1 in an airtight manner at least along fixing portions 16. Via a valve-type connecting means 30, air can be conducted away out of the covered region of the wound 3 by means of a hose line 32 by a vacuum source (not shown). As a result, a vacuum is adjusted in the covered wound region. By means of said pressure, it is effected that wound secretions enter the absorbent body 12 through the woven-fabric-type separating element 20. As a result, the wound secretions can no longer damage healthy body tissue and impede the wound-healing process. The wound secretions can remain in the absorbent body 12 or be conducted away out of the covered wound region as a whole by means of the hose line 32.

One embodiment of a separating element 20 according to the invention is shown in FIG. 2. The separating element 20, which is preferably a woven fabric, is applied to a film-type carrier element 28 by means of two strip-shaped adhesive layers 24 along the long sides of the separating element 20. In this case, the separating element 20 and the carrier element 28 are designed to be substantially transparent so as to be able to be cut to size as required directly in the region of the wound. The thin separating element 20 and the likewise thin carrier element 28 can be precisely cut to size together using conventional scissors. After the cutting to size, the thin and sensitive separating element 20 can be removed from the film-type carrier element 28, the adhesive layers 24 remaining on the separating element 20. The adhesive layers 24 of the separating element 20 can then be applied to the wound region, the adhesive layers 24 being used for fixing to the skin region 5 at the edge of the wound 3.

1.-10. (canceled)

11. A dressing arrangement for treating a wound of a human or animal body by means of vacuum, comprising
an absorbent body for absorbing wound secretions, the absorbent body being designed for insertion in a cavity of the wound,
a separating element, which is arranged between the wound and the absorbent body, the separating element having a woven fabric,
a cover element, which is designed to cover the wound, together with the absorbent body, in a substantially airtight manner, and
a connecting means which is arranged for connecting a vacuum source to the cover element,
the separating element is designed to be larger than the wound to be treated, a protruding edge portion being formed, and
the edge portion is arranged on a skin region at the edge of the wound,
the separating element being designed to be bio-compatible and anti-adhesive,
wherein
the woven fabric is formed of mono-filament threads having a smooth surface and a thread diameter of between 10 and 100 μm,
the woven fabric is provided with a mesh size of from 40 to 500 μm, and
at the edge portion, an adhesive layer is arranged at least in some regions to adhere the separating element to the skin region.

12. The dressing arrangement according to claim 11,
wherein
the mono-filament threads have a thread diameter of between 30 and 50 μm.

13. The dressing arrangement according to claim 11,
wherein
the woven fabric is provided with a mesh size of from 70 to 300 μm.

14. The dressing arrangement according to claim 11,
wherein
the cover element is designed to be larger than the separating element.

15. The dressing arrangement according to claim 11,
wherein
the separating element is detachably arranged on a carrier element.

16. The dressing arrangement according to claim 15,
wherein
the carrier element is a carrier film that can be cut to size.

17. A method for treating a wound of a human or animal body, in particular by means of the dressing arrangement according to claim 11,
an absorbent body being inserted in a cavity of the wound to absorb wound secretions,
a separating element being arranged between the wound and the absorbent body,
the wound, together with the inserted absorbent body, being covered by means of a cover element in a substantially airtight manner, and
by a connecting means, a vacuum source being connected to the cover element, by means of which source a vacuum in the covered wound is adjusted,
wherein
the separating element is designed to be larger than the wound to be treated, a protruding edge portion being formed,
the edge portion is arranged on a skin region at the edge of the wound, and
an adhesive layer of the separating element is adhered to the edge portion on the skin region.

18. The method according to claim 17,
wherein
the separating element is detachably attached to a carrier element which can be cut to size and, before application to the wound, is cut to the intended size together with the carrier element.
